(19) United States
(12) Patent Application Publication
(10) Pub. No.: US 2016/0038454 A1
(43) Pub. Date: Feb. 11, 2016

(54) INTRAVENOUS FORMULATIONS OF TRIPTOLIDE COMPOUNDS AS IMMUNOMODULATORS AND ANTICANCER AGENTS

(71) Applicants: PHARMAGENESIS, INC., Redwood City,, CA (US); MYELORX, LLC, Vallejo, CA (US)

(72) Inventors: Jinhua AN, Santa Clara, CA (US); Weiguo HE, Beijing (CN); John M. Fidler, Oakland, CA (US); John H. Musser, San Carlos, CA (US)

(21) Appl. No.: 14/776,606

(22) PCT Filed: Mar. 15, 2014
(86) PCT No.: PCT/US14/30041

§ 371 Date: Sep. 14, 2015

Related U.S. Application Data
(60) Provisional application No. 61/794,926, filed on Mar. 15, 2013.

(51) Int. Cl. A61K 31/343; A61K 47/14; A61K 31/365; A61K 47/28; A61K 47/44; A61K 47/24; A61K 47/10

(52) U.S. Cl. CPC A61K 31/343; A61K 47/24; A61K 47/14; A61K 47/10; A61K 47/28; A61K 47/44; A61K 31/365

(57) ABSTRACT

Intravenous formulations of triptolide and triptolide derivatives are disclosed for use in immunomodulation and anti-proliferative agents.

### TECHNICAL FIELD

The present disclosure is directed to formulations of triptolide-derived compounds, useful as immunomodulators, anti-inflammatory and anticancer agents.

### BACKGROUND

Immunosuppressive agents are widely used in the treatment of autoimmune disease and in treating or preventing transplantation rejection, including the treatment of graft-versus-host disease (GVHD), a condition in which transplanted (grafted) cells attack the recipient (host) cells. Common immunosuppressive agents include azathioprine, corticosteroids, cyclophosphamide, methotrexate, 6-mercaptopurine, vincristine, and cyclosporin A. In general, none of these drugs are completely effective, and most are limited by severe toxicity. For example, cyclosporin A, a widely used agent, is significantly toxic to the kidney. In addition, doses needed for effective treatment may increase the patient's susceptibility to infection by a variety of opportunistic invaders.

The compound triptolide, obtained from the Chinese medicinal plant Tripterygium wilfordii (TW), and certain derivatives and prodrugs thereof, have been identified as having significant immunosuppressive activity. Various prodrugs and other analogs of triptolide have also shown such activity. See, for example, U.S. Pat. Nos. 4,005,108; 5,294,443; 5,648,376; 5,663,335; 5,759,550; 5,843,452; 5,962,516 and 6,150,539, each of which is incorporated herein by reference in its entirety. Triptolide and certain derivatives/analogs and prodrugs thereof have also been reported to show significant anticancer activity, including reduction of solid tumors in vivo; see, for example, Kupchan et al., J. Am. Chem. Soc. 94:7194 (1972), as well as co-owned U.S. Pat. No. 6,620,843, also incorporated by reference, herein, in its entirety. Triptolide and its prodrugs and other analogs have also shown significant anticancer activity, including reduction of solid tumors in vivo. See, for example, co-owned U.S. Pat. No. 6,620,843, which is incorporated herein by reference in its entirety, see, for example, Fidler et al., Mol. Cancer Ther. 2(9):855-62 (2003).

The analog can be designated a “selectively binding” analog if its binding affinity to a given first target molecule differs from its binding affinity to a second target molecule by a factor of 10 or more.

Although derivatives and prodrugs of triptolide have provided benefits relative to native triptolide in areas such as pharmacokinetics or biodistribution, e.g. by virtue of differences in lipid or aqueous solubility, or via their activity as prodrugs, the biological activity per se of triptolide derivatives is often significantly less than that of native triptolide.

The foregoing examples of the related art and limitations related therewith are intended to be illustrative and not exclusive. Other limitations of the related art will become apparent to those of skill in the art upon a reading of the specification and a study of the drawings.

### BRIEF DESCRIPTION OF THE DRAWINGS

FIG. 1: Comparison of plasma triptolide concentrations over time upon injection of the prodrug PG796(MRx102) vs. triptolide

### BRIEF SUMMARY

Examples of the related art and limitations related therewith, as set forth herein, are intended to be illustrative and not exclusive. Other limitations of the related art will become apparent to those of skill in the art upon a reading of the specification and a study of the drawings.

In one aspect, a composition is provided for intravenous administration of an emulsion comprising triptolide or a triptolide derivative having a clogP of 0.5 or higher, the emulsion comprising (a) 15 to 45% by weight lipid, (b) 0 to 50% by weight of a medium chain triglyceride, (c) 0.5 to 3% by weight phospholipid, (d) 0 to 5% by weight of glycerin, (e) 0.1 to 0.3% by weight of a sodium cholate, (f) about 50 to 60% by weight water, and (g) about 0.5 to about 3 mg/mL triptolide or a triptolide derivative. In some embodiments, no glycerin is used. In some embodiments, the concentration of triptolide or triptolide derivative is about 0.5 mg/mL to about 3 mg/mL. In some embodiments, the concentration of triptolide or triptolide derivative is about 1 mg/mL to about 2 mg/mL.

In some embodiments, the composition comprises 15 to 45% by weight lipid, wherein the lipid is selected from the group consisting of soybean oil, castor oil, corn oil, cottonseed oil, olive oil, peanut oil, peppermint oil, safflower oil, sesame oil, coconut oil or palm seed oil.

In some embodiments, the medium chain triglyceride is 20% by weight and is selected from the group consisting of glyceryl trioctanoate, glyceryl trihexanoate, glyceryl triheptanoate, glyceryl trinonanoate and glyceryl tridecanoate.

In some embodiments, the phospholipid is selected from the group consisting of hydrogenated soy phosphatidylcholine, di stearoylphosphatidylglycerol, L-alpha-dimyristoylphosphatidylcholine and L-alpha-dimyristoylphosphatidylglycerol.

In some embodiments, the glycerin is selected from the group consisting of polyethylene glycol 300, polyethylene glycol 400, ethanol, propylene glycol, N-methyl-2-pyrrolidone, dimethylacetamide, and dimethylsulfoxide.

In some embodiments, the sodium cholate is selected from the group consisting of sodium taurocholate, sodium tauro-β-muricholate, sodium taurodeoxycholate, sodium taurochenodeoxycholate, sodium glycocholate, sodium glycodeoxycholate and sodium glycochenodeoxycholate.

In some embodiments, the composition for intravenous administration of an emulsion comprising triptolide or a triptolide derivative having a clogP of 0.5 or higher, is an emulsion comprising (a) 15 to 45% by weight lipid, (b) 0 to 95% by weight of a medium chain triglyceride, (c) 0.5 to 3% by weight phospholipid, (d) 0 to 5% by weight of glycerin, (e) 0.1 to 0.3% by weight of a sodium cholate, and (f) about 0.5 to about 3 mg/mL triptolide or a triptolide derivative, and is stored as an anhydrous mixture, and an aqueous solution is added prior to administration.

In some embodiments, composition for oral administration of an emulsion comprising triptolide or a triptolide derivative having a clogP of 0.5 or higher, is an emulsion comprising (a) 15 to 45% by weight lipid, (b) 0 to 95% by weight of a medium chain triglyceride, (c) 0.5 to 3% by weight phospholipid, (d) 0 to 5% by weight of glycerin, (e) 0.1 to 0.3% by weight of a sodium cholate, and (f) about 0.5 to about 3 mg/mL triptolide or a triptolide derivative, and is stored as an anhydrous mixture, and an aqueous solution is added prior to administration.

In one aspect, a composition for oral administration of an emulsion comprising triptolide or a triptolide derivative having a clogP of 0.5 or higher is provided.

In some embodiments, the composition comprises a triptolide derivative selected from the group consisting of compounds according to structure I. In some embodiments, the composition comprises a triptolide derivative selected from the group consisting of compounds according to structure II. In some embodiments, the composition comprises a triptolide derivative selected from the group consisting of compounds according to structure III. In some embodiments, the composition comprises a triptolide derivative selected from the group consisting of compounds according to structure IV.

In one aspect, a method is provided for effecting immunosuppression, immunomodulation or inhibiting cell proliferation, wherein the method comprises intravenously administering an emulsion comprising triptolide or a triptolide derivative having a clogP of 0.5 or higher to a subject in need in an amount effective for immunosuppression, immunomodulation or inhibiting cell proliferation.

In one aspect, a method is provided for inducing apoptosis in a cell, wherein the method comprises intravenously administering an emulsion comprising triptolide or a triptolide derivative having a clogP of 0.5 or higher to a subject in need in an amount effective for inducing apoptosis.

Additional embodiments of the present methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### I. DEFINITIONS

As used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

Where a range of values is provided, it is intended that each intervening value, to the tenth of the unit of the lower limit unless the context clearly dictates otherwise, between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

“Alkyl” refers to a saturated acyclic monovalent radical containing carbon and hydrogen, which may be linear or branched. Examples of alkyl groups are methyl, ethyl, n-butyl, t-butyl, n-heptyl, and isopropyl. “Cycloalkyl” refers to a fully saturated cyclic monovalent radical containing carbon and hydrogen, which may be further substituted with alkyl. Examples of cycloalkyl groups are cyclopropyl, methyl cyclopropyl, cyclobutyl, cyclopentyl, ethylcyclopentyl, and cyclohexyl. “Lower alkyl” refers to such a group having one to six carbon atoms, and in some embodiments one to four carbon atoms.

“Alkenyl” refers to an acyclic monovalent radical containing carbon and hydrogen, which may be linear or branched, and which contains at least one carbon-carbon double bond (C═C). “Alkynyl” refers to an acyclic monovalent radical containing carbon and hydrogen, which may be linear or branched, and which contains at least one carbon-carbon triple bond (C≡C). “Lower alkenyl” or “lower alkynyl” such a group having two to six carbon atoms, and in some embodiments two to four carbon atoms.

“Acyl” refers to a radical having the form —(C═O)R, where R is alkyl (alkylacyl) or aryl (arylacyl). “Acyloxy” refers to a group having the form —O(C═O)R.

“Aryl” refers to a monovalent aromatic radical having a single ring (e.g., benzene) or two condensed rings (e.g., naphthyl). As used herein, aryl is a monocyclic and carbocyclic (non-heterocyclic), e.g. a benzene (phenyl) ring or substituted benzene ring. By “substituted” is meant that one or more ring hydrogens is replaced with a group such as a halogen (e.g. fluorine, chlorine, or bromine), lower alkyl, nitro, amino, lower alkylamino, hydroxy, lower alkoxy, or halo(lower alkyl).

“Arylalkyl” refers to an alkyl, often lower (C₁-C₄, or C₁-C₂) alkyl, substituent which is further substituted with an aryl group; examples are benzyl and phenethyl.

A “heterocycle” refers to a non-aromatic ring, often a 5- to 7-membered ring, whose ring atoms are selected from the group consisting of carbon, nitrogen, oxygen and sulfur. In some embodiments, the ring atoms include 3 to 6 carbon atoms. Such heterocycles include, for example, pyrrolidine, piperidine, piperazine, and morpholine.

“Halogen” or “halo” refers to fluorine, chlorine, bromine, or iodine.

For the purposes of the current disclosure, the following numbering scheme is used for triptolide and triptolide derivatives:

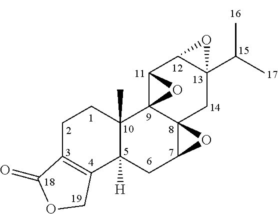 [SMILES: CC(C)[C@@]12C[C@]34O[C@H]3C[C@H]3C5=C(CC[C@]3(C)[C@@]43O[C@H]3[C@@H]1O2)C(=O)OC5]

### II. TRIPTOLIDE ANALOGS

Triptolide analogs, as the term is used herein, include various structural modifications of the natural product triptolide (designated herein as PG490). They may include naturally occurring analogs, such as 2-hydroxytriptolide or 16-hydroxytriptolide (tripdiolide), although the term generally refers herein to synthetically prepared analogs. As used herein, the term “triptolide-related compounds” refers to triptolide and its analogs, and preferably refers to analogs.

Structural modifications may include, for example, ring opening of an epoxy or lactone ring of triptolide; conversion of a hydroxyl group (either naturally occurring or produced by such ring opening) to a carboxylic ester, inorganic ester (e.g. sulfonate), carbonate, or carbamate, to an aldehyde or ketone via oxidation, or to a hydrogen atom via subsequent reduction; conversion of a single bond to a double bond, and/or substitution of a hydrogen atom by a halogen, alkyl, alkenyl, hydroxyl, alkoxy, acyl, or amino group. Examples of triptolide analogs have been described in several US patents, including U.S. Pat. Nos. 5,663,335, 6,150,539, 6,458,537, and 6,569,893, each of which is hereby incorporated by reference in its entirety. The compounds can be prepared, as described therein, from triptolide, a plant-derived diterpene triepoxide. Triptolide and its analogs have shown beneficial immunosuppressive and cytotoxic activity, as described, for example, in the above-referenced patents.

Exemplary triptolide analogs include 14-methyltriptolide (designated PG670; see US application pubn. no. 20040152767), triptolide 14-tert-butyl carbonate (designated PG695; see PCT Pubn. No WO 2003/101951), 14-deoxy-14α-fluoro triptolide (designated PG763; see U.S. Provisional Appn. Ser. No. 60/449,976), triptolide 14-(α-dimethylamino)acetate (designated PG702; see U.S. Pat. No. 5,663,335), 5-α-hydroxy triptolide (designated PG701; see U.S. Provisional Appn. Ser. No. 60/532,702), 19-methyl triptolide (designated PG795; see U.S. Provisional Appn. Ser. No. 60/549,769), and 18-deoxo-19-dehydro-18-benzoyloxy-19-benzoyl triptolide (designated PG796; see U.S. Provisional Appn. Ser. No. 60/549,769). Each of these applications and publications is hereby incorporated by reference in its entirety.

Many of these compounds are believed to act as prodrugs, by converting in vivo to triptolide, as observed for PG490-88, above. Others, such as 14-deoxy-14α-fluoro triptolide (PG763), are not expected to undergo such conversion, but nonetheless exhibit biological activities shown by triptolide (e.g. cytotoxicity in human T cell lymphoma (Jurkat) cells and inhibition of IL-2), as reported in U.S. application Ser. No. 60/449,976, cited above.

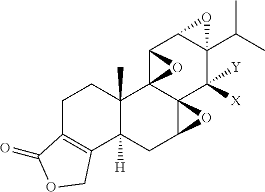

### Exemplary Triptolide Derivatives and Prodrugs

TABLE 1

| compound | X | Y |
|---|---|---|
| PG490-88 | —O(CO)CH₂CH₂COOH | —H |
| PG670 | —OH | —CH₃ |
| PG695 | —O(CO)OC(CH₃)₃ | —H |
| PG702 | —O(CO)CH₂N(CH₃)₂ | —H |
| PG673 | —H | —F |

Triptolide analogs for screening can be generated by combinatorial chemistry or other type of preparation to generate diversity of chemical structure or substituents.

The active ingredient in the formulation is triptolide or a derivative of triptolide, as described below. For example, the disclosure provides compounds of structure I:

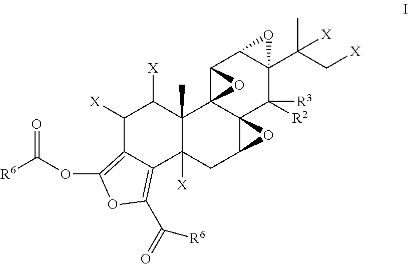

where

each R⁶ is independently selected from alkyl, alkenyl, alkynyl, or aryl;

CR²R³ is CHOH or C═O;

at most one of the groups X is hydroxyl, and the remaining groups X are hydrogen.

In some embodiments of structure I, CR²R³ is CHOH, often having the β-hydroxy configuration. In some embodiments, each X is hydrogen; however, in selected embodiments, exactly one of the indicated groups X is hydroxyl. Locations for hydroxyl substitution often include carbons 2 and 16, as shown in the numbering scheme above.

In some embodiments, each said alkyl, alkenyl, and alkynyl moiety present in a compound of structure I includes at most four carbon atoms, and each said aryl moiety is monocyclic and non-heterocyclic; e.g. substituted or unsubstituted phenyl.

In selected embodiments of structure I, each R⁶ is aryl; often, each R⁶ is phenyl. This includes the compound designated herein as PG796, where each R⁶ is unsubstituted phenyl.

The disclosure also provides compounds represented by structure II:

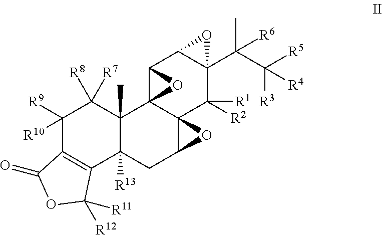

where:
CR¹R² is selected from CHOH, C═O, CHF, CF₂ and C(CF₃)OH;
CR⁶ and CR¹³ are selected from CH, COH and CF;
CR⁷R⁸, CR⁹R¹⁰ and CR¹¹R¹² are selected from CH₂, CHOH, C═O, CHF and CF₂; and
CR³R⁴R⁵ is selected from CH₃, CH₂OH, C═O, COOH, CH₂F, CHF₂ and CF₃,
such that: at least one of R¹-R¹³ comprises fluorine;
no more than two, and often no more than one, of CR³R⁴R⁵, CR⁶, CR⁷R⁸, CR⁹R¹⁰, CR¹¹R¹², and CR¹³ comprises fluorine or oxygen;
and, when CR¹R² is CHOH, CR³R⁴R⁵ is not CH₂F.

In some embodiments, the stereochemistry at CR⁷R⁸ is such that, when CR⁷R⁸ is CHOH, it has a β-hydroxy configuration, and, when CR⁷R⁸ is CHF, it has an α-fluoro configuration. Similarly, the stereochemistry at CR⁹R¹⁰ is often such that, when CR⁹R¹⁰ is CHOH, it has a β-hydroxy configuration, and, when CR⁹R¹⁰ is CHF, it has an α-fluoro configuration.

In some embodiments of structure II, CR¹R² is CHF, having an α-fluoro configuration.

Some embodiments also include compounds in which exactly one carbon center selected from CR¹R², CR³R⁴R⁵, CR⁶, CR⁷R⁸, CR⁹R¹⁰, and CR¹¹R¹² comprises fluorine. In some embodiments, exactly one of CR¹R², CR⁶, CR⁷R⁸, CR⁹R¹⁰, and CR¹¹R¹² comprises fluorine.

In selected embodiments, only CR¹R² comprises fluorine. Accordingly, in these embodiments, CR¹R² is selected from CF₂, CHF, and C(CF₃)OH. The stereochemistry at CR¹R² is such that, when CR¹R² is C(CF₃)OH, it has a β-hydroxy configuration, and, when CR¹R² is CHF, it has an α-fluoro configuration. In selected embodiments of structure II, the compound is PG763.

In other selected embodiments of structure II, either CR⁹R¹⁰ or CR³R⁴R⁵ comprises fluorine, and CR¹R² comprises oxygen; for example, CR¹R² is C═O or, in some embodiments, CHOH (β-hydroxy). In these embodiments, for example, CR⁹R¹⁰ is selected from CF₂ and CHF (e.g., α-fluoro), or CR³R⁴R⁵ is selected from CHF₂ or CF₃.

In further selected embodiments of structure II, either CR⁷R⁸ or CR¹¹R¹² (comprises fluorine, and CR¹R² comprises oxygen; for example, CR¹R² is C═O or, in some embodiments, CHOH (β-hydroxy). In these embodiments, for example, CR⁷R⁸ is selected from CF₂ and CHF (e.g., α-fluoro), or CR¹¹R¹² is selected from CF₂ and CHF.

The disclosure also provides compounds represented by structure III

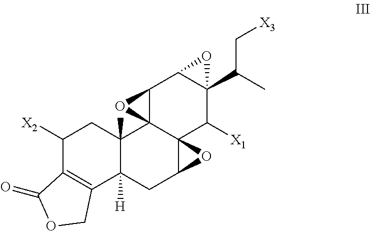

In the structure III, the variables are defined as follows:

X¹ is OH or OR¹, and X² and X³ are independently OH, OR¹ or H, with the proviso that at least one of X¹, X² and X³ is OR¹, and at least one of X² and X³ is H; and

OR¹ is O—(C═O)—Z, where Z is selected from the group consisting of: —OR², —O—Y—(C═O)—OR³, —O—Y—NR⁴R⁵, —NR⁴R⁵, —NR³—Y—(C═O)—OR³, and —NR³—Y—NR⁴N⁵;

wherein

Y is a divalent alkyl, alkenyl or alkynyl group having up to six carbon atoms;

R² is selected from alkyl, alkenyl, alkynyl, cycloalkyl, cycloalkenyl, aryl, aralkyl, hydroxyalkyl, alkoxyalkyl, aryloxyalkyl, and acyloxyalkyl;

each R³ is independently selected from hydrogen and R²; and

R⁴ and R⁵ are independently selected from hydrogen, alkyl, alkenyl, alkynyl, cycloalkyl, cycloalkenyl, aryl, aralkyl, hydroxyalkyl, alkoxyalkyl, aryloxyalkyl, and acyloxyalkyl, or R⁴ and R⁵ taken together form a 5- to 7-member heterocyclic ring whose ring atoms are selected from the group consisting of carbon, nitrogen, oxygen and sulfur, wherein the ring atoms include at most 3 heteroatoms.

The groups defined as R², R³, R⁴, and R⁵, when selected from alkyl, alkenyl, and alkynyl, can have up to six carbon atoms. When selected from cycloalkyl or cycloalkenyl, they often have 3 to 7, or, for cycloalkenyl, 5 to 7 carbon atoms. When selected from aralkyl, hydroxyalkyl, alkoxyalkyl, aryloxyalkyl, and acyloxyalkyl, the alkyl components of these groups often have up to six carbon atoms. In one embodiment, each of these groups is independently selected from alkyl, aryl, aralkyl, and alkoxyalkyl.

In selected embodiments of structure III, X²═X³═H, and Y is —CH₂— or —CH₂CH₂—. In further embodiments, OR¹ is selected from the group consisting of O—(C═O)—OR², O—(C═O)—O—Y—(C═O)—OR³, and O—(C═O)—O—Y—NR⁴R⁵ (carbonate derivatives). In other embodiments, OR¹ is -selected from the group consisting of O—(C═O)—NR⁴R⁵, O—(C═O)—NR³⁻Y—(C═O)—OR³, and O—(C═O)—NR³—Y—NR⁴N⁵ (carbamate derivatives). In selected embodiments of structure III, the compound is PG695.

The disclosure also provides compounds represented by structure IV.

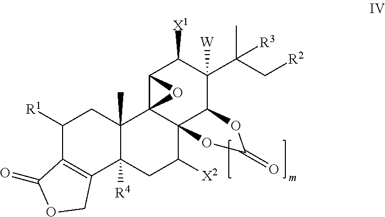

where
each of R¹, R², R³, and R⁴ is independently selected from hydrogen, hydroxyl, —O(CO)ₙX, —O(CO)ₙOR⁵, and —O(CO)ₙN(R⁵)₂, where X is halogen, R⁵ is hydrogen or lower alkyl, and n is 1-2,
with the proviso that at least three of R¹, R², R³, and R⁴ are hydrogen;
m is 1-2;
X² is halogen, such as F or Cl; and
X¹ is halogen, often Cl, and W is hydroxyl; or X¹ and W together form an epoxy group.

When any of each of R¹, R², R³, and R⁴ is selected from —O(CO)ₙX, —O(CO)ₙOR⁵, or —O(CO)ₙN(R⁵)₂, the variable n is often 1.

In selected embodiments of structure IV, each of R¹, R², R³, and R⁴ is hydrogen. In further selected embodiments, m=1. In selected embodiments of structure IV, the compound is PG762.

### III. BIOLOGICAL ACTIVITY

The cytotoxic activity of a compound according to structure I, 18-deoxo-19-dehydro-18-benzoyloxy-19-benzoyl triptolide (designated PG796), can be evaluated using a standard MTT assay, as described in Example 3 and the immunosuppressive activity of these compounds was evaluated in a standard IL-2 inhibition assay, as described in Example 4. PG796 showed a higher level of activity in both assays than the known prodrug, triptolide 14-succinate (designated PG490-88). In particular, triptolide 14-succinate incubated in human serum was much less active in these assays than triptolide 14-succinate incubated in mouse serum, while PG796 showed high, and essentially equivalent, activity in both cases. (Incubation is expected to convert triptolide 14-succinate to triptolide and PG796 to the monoderivatized compound, 19-benzoyl triptolide, shown in the above synthetic scheme.)

The cytotoxic activity of three compounds of structure IV, designated PG757, PG762 and PG830, and one additional compound designated PG782, can be evaluated using a standard MTT assay as described in Example 2. The immunosuppressive activity of these compounds was evaluated in a standard IL-2 inhibition assay as described in Example 3.

The compound PG757 incubated in serum was significantly more cytotoxic in the MTT assay than triptolide; see Table 2 below. (The data for test compounds in Table 2 is for compounds incubated in serum for 24 hrs.) Incubated PG782 was also more potent than triptolide, and incubated PG762 was of comparable potency. Several test compounds, when incubated in serum, were comparable to triptolide in suppression of IL-2.

TABLE 2

| | Viability/Cytotoxicity | Immunosuppression |
|---|---|---|
| Compound | MTT (ED₅₀) | IL-2 (IC₅₀) |
| PG490 | 60 nM | 4 nM |
| (triptolide) | | |
| PG757 | 32 nM | 9 nM |
| PG762 | 60 nM | 9 nM |
| PG782 | 53 nM | 2 nM |

Incubation in serum converts prodrug compounds to triptolide, and this has been shown to happen within about 5 minutes for PG757 and PG762.

### IV. THERAPEUTIC COMPOSITIONS

Formulations containing the triptolide derivatives of the disclosure may take the form of solid, semi-solid, lyophilized powder, or liquid dosage forms, such as tablets, capsules, powders, sustained-release formulations, solutions, suspensions, emulsions, ointments, lotions, or aerosols, and in some embodiments in unit dosage forms suitable for simple administration of precise dosages. The compositions typically include a conventional pharmaceutical carrier or excipient and may additionally include other medicinal agents, carriers, or adjuvants.

In some embodiments, the composition will be about 0.5% to 75% by weight of a compound or compounds of the disclosure, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

The composition may be administered to a subject orally, transdermally or parenterally, e.g., by intravenous, subcutaneous, intraperitoneal, or intramuscular injection. For use in oral liquid preparation, the composition may be prepared as a solution, suspension, emulsion, or syrup, being supplied either in liquid form or a dried form suitable for hydration in water or normal saline. For parenteral administration, an injectable composition for parenteral administration will typically contain the triptolide derivative in a suitable intravenous solution, such as sterile physiological salt solution.

Liquid compositions can be prepared by dissolving or dispersing the triptolide derivative (about 0.5% to about 20%) and optional pharmaceutical adjuvants in a pharmaceutically acceptable carrier, such as, for example, aqueous saline, aqueous dextrose, glycerol, or ethanol, to form a solution or suspension.

The compound may also be administered by inhalation, in the form of aerosol particles, either solid or liquid, often of respirable size. Such particles are sufficiently small to pass through the mouth and larynx upon inhalation and into the bronchi and alveoli of the lungs. In general, particles ranging from about 1 to 10 microns in size, and often less than about 5 microns in size, are respirable. Liquid compositions for inhalation comprise the active agent dispersed in an aqueous carrier, such as sterile pyrogen free saline solution or sterile pyrogen free water. If desired, the composition may be mixed with a propellant to assist in spraying the composition and forming an aerosol.

Methods for preparing such dosage forms are known or will be apparent to those skilled in the art; for example, see Remington's Pharmaceutical Sciences (19th Ed., Williams & Wilkins, 1995). The composition to be administered will contain a quantity of the selected compound in an effective amount for effecting immunosuppression in a subject or apoptosis in a targeted cell.

As described, for example, in Panchagnula et al. (2000), the partition coefficient or logP of a pharmaceutical agent can affect its suitability for various routes of administration, including oral bioavailability. The compounds described herein, by virtue of substitution of fluorine for one or more hydroxyl groups, are expected to have higher calculated logP values than the parent compound, triptolide, making them better candidates for oral availability.

The lipid formulations disclosed herein are useful for intravenous administration, as well as for oral administration. Lipid and surfactant based formulations are well recognized as a feasible approach to improve oral bioavailability of poorly soluble compounds. Several drug products utilizing lipid and surfactant based formulations and intended for oral administration are commercially available. For example, Sandimmune® and Sandimmune, Neoral® (cyclosporin A, Novartis), Norvir® (ritonavir), and Fortovase® (saquinavir) have been formulated in self-emulsifying drug delivery systems. Indeed, a recent review summarizes published pharmacokinetic studies of orally administered lipid based formulations of poorly aqueous soluble drugs in human subjects. (Fatouros, et al., (2007) Therapeutics and Clinical Risk Management 3(4):591-604).

### V. IMMUNOMODULATING AND ANTIINFLAMMATORY TREATMENT

A compound according to structure I, 18-deoxo-19-dehydro-18-benzoyloxy-19-benzoyl triptolide (designated PG796), inhibited IL-2 production in Jurkat cells (see Example 3) in a dose-dependent manner. The disclosure thus includes the use of the formulations containing an active ingredient(s) as immunosuppressive agents, e.g. as an adjunct to transplant procedures or in treatment of autoimmune disease.

One utility envisioned for this disclosure is for the treatment of human diseases of the immune system regulatory abnormalities. Immunoregulatory abnormalities have been shown to exist in a wide variety of autoimmune and chronic inflammatory diseases, including systemic lupus erythematosis, chronic rheumatoid arthritis, type I and II diabetes mellitus, inflammatory bowel disease, biliary cirrhosis, uveitis, multiple sclerosis and other disorders such as Crohn's disease, ulcerative colitis, pemphigus, bullous pemphigoid, sarcoidosis, psoriasis, ichthyosis, Graves ophthalmopathy, Grave's disease and asthma. Although the underlying pathogenesis of each of these conditions may be quite different, they have in common the appearance of a variety of autoantibodies and self-reactive lymphocytes. Such self-reactivity may be due, in part, to a loss of the homeostatic controls under which the normal immune system operates.

Similarly, following a bone-marrow transplant or other transplant of hematopoietic stem cells from a donor tissue source containing mature lymphocytes, the transferred lymphocytes recognize the host tissue antigens as foreign. These cells become activated and mount an attack upon the host (a graft-versus-host response) that can be life-threatening. Moreover, following an organ transplant, the host lymphocytes recognize the foreign tissue antigens of the organ graft and mount cellular and antibody-mediated immune responses (a host-versus-graft response) that lead to graft damage and rejection.

One result of an autoimmune or a rejection reaction is tissue destruction caused by inflammatory cells and the mediators they release. Anti-inflammatory agents such as NSAIDs act principally by blocking the effect or secretion of these mediators but do nothing to modify the immunologic basis of the disease. On the other hand, cytotoxic agents, such as cyclophosphamide, act in such a nonspecific fashion that both the normal and autoimmune responses are shut off. Indeed, patients treated with such nonspecific immunosuppressive agents are as likely to succumb from infection as they are from their autoimmune disease.

The compositions of the present disclosure are useful in applications for which triptolide and its prodrugs and other derivatives have proven effective, e.g. in immunosuppression therapy, as in treating an autoimmune disease, preventing transplantation rejection, or treating or preventing graft-versus-host disease (GVHD). See, for example, co-owned U.S. Pat. No. 6,150,539, which is incorporated herein by reference. Triptolide and the present derivatives are also useful for treatment of other inflammatory conditions, such as traumatic inflammation, and in reducing male fertility.

The compositions are useful for inhibiting rejection of a solid organ transplant, tissue graft, or cellular transplant from an incompatible human donor, thus prolonging survival and function of the transplant, and survival of the recipient. This use would include, but not be limited to, solid organ transplants (such as heart, lung, pancreas, limb, muscle, nerve, kidney and liver), tissue grafts (such as skin, corneal, intestinal, gonadal, bone, and cartilage), and cellular transplants (e.g., cells from pancreas such as pancreatic-islet cells, brain and nervous tissue, muscle, skin, bone, cartilage and liver) including xenotransplants, etc.

The compositions are also useful for inhibiting xenograft (interspecies) rejection; i.e. in preventing the rejection of a solid organ transplant, tissue graft, or cellular transplant from a non-human animal, whether natural in constitution or bioengineered (genetically manipulated) to express human genes, RNA, proteins, peptides or other non-native, xenogeneic molecules, or bioengineered to lack expression of the animal's natural genes, RNA, proteins, peptides or other normally expressed molecules. The disclosure also includes the use of a composition as described above to prolong the survival of such a solid organ transplant, tissue graft, or cellular transplant from a non-human animal.

Also included are methods of treatment of autoimmune diseases or diseases having autoimmune manifestations, such as Addison's disease, autoimmune hemolytic anemia, autoimmune thyroiditis, Crohn's disease, diabetes (Type I, juvenile-onset or recent-onset diabetes mellitus), Graves' disease, Guillain-Barre syndrome, systemic lupus erythematosis (SLE), lupus nephritis, multiple sclerosis, myasthenia gravis, psoriasis, primary biliary cirrhosis, rheumatoid arthritis, uveitis, asthma, atherosclerosis, Hashimoto's thyroiditis, allergic encephalomyelitis, glomerulonephritis, and various allergies.

Further uses may include the treatment and prophylaxis of inflammatory and hyperproliferative skin diseases and cutaneous manifestations of immunologically mediated illnesses, such as psoriasis, atopic dermatitis, pemphigus, urticaria, cutaneous eosinophilias, acne, and alopecia areata; various eye diseases such as conjunctivitis, uveitis, keratitis, and sarcoidosis; inflammation of mucous and blood vessels such as gastric ulcers, vascular damage caused by ischemic diseases and thrombosis, ischemic bowel diseases, inflammatory bowel diseases, and necrotizing enterocolitis; intestinal inflammations/allergies such as Coeliac diseases, Crohn's disease and ulcerative colitis; renal diseases such as interstitial nephritis, Good-pasture's syndrome, hemolytic-uremic syndrome and diabetic nephropathy; hematopoietic diseases such as idiopathic thrombocytopenia purpura and autoimmune hemolytic anemia; skin diseases such as dermatomyositis and cutaneous T cell lymphoma; circulatory diseases such as arteriosclerosis and atherosclerosis; nephrotic syndrome such as glomerulonephritis; renal diseases such as ischemic acute renal insufficiency and chronic renal insufficiency; and Behcet's disease.

The compositions and method of the disclosure are also useful for the treatment of inflammatory conditions such as asthma, both intrinsic and extrinsic manifestations, for example, bronchial asthma, allergic asthma, intrinsic asthma, extrinsic asthma and dust asthma, particularly chronic or inveterate asthma (for example, late asthma and airway hyperresponsiveness), or other lung diseases including allergies and reversible obstructive airway disease, including bronchitis and the like. The composition and method may also be used for treatment of other inflammatory conditions, including traumatic inflammation, inflammation in Lyme disease, chronic bronchitis (chronic infective lung disease), chronic sinusitis, sepsis associated acute respiratory distress syndrome, and pulmonary sarcoidosis. For treatment of respiratory conditions such as asthma, the composition is often administered via inhalation, but any conventional route of administration may be useful.

In treating an autoimmune condition, the patient is given the composition on a periodic basis, e.g., 1-2 times per week, at a dosage level sufficient to reduce symptoms and improve patient comfort. For treating rheumatoid arthritis, in particular, the composition may be administered by intravenous injection or by direct injection into the affected joint. The patient may be treated at repeated intervals of at least 24 hours, over a several week period following the onset of symptoms of the disease in the patient. The dose that is administered is often in the range of 1-25 mg/kg patient body weight per day, often in lower amounts for parenteral administration, and higher amounts for oral administration. Optimum dosages can be determined by routine experimentation according to methods known in the art.

For therapy in transplantation rejection, the method is intended particularly for the treatment of rejection of heart, kidney, liver, cellular, and bone marrow transplants, and may also be used in the treatment of GVHD. The treatment is typically initiated perioperatively, either soon before or soon after the surgical transplantation procedure, and is continued on a daily dosing regimen, for a period of at least several weeks, for treatment of acute transplantation rejection. During the treatment period, the patient may be tested periodically for immunosuppression level, e.g., by a mixed lymphocyte reaction involving allogeneic lymphocytes, or by taking a biopsy of the transplanted tissue.

In addition, the composition may be administered chronically to prevent graft rejection, or in treating acute episodes of late graft rejection. As above, the dose administered is often 1-25 mg/kg patient body weight per day, with lower amounts for parenteral administration, and higher amounts for oral administration. The dose may be increased or decreased appropriately, depending on the response of the patient, and over the period of treatment, the ability of the patient to resist infection.

In treatment or prevention of graft-versus-host disease, resulting from transplantation into a recipient of matched or mismatched bone marrow, spleen cells, fetal tissue, cord blood, or mobilized or otherwise harvested stem cells, the dose is often in the range 0.25-2 mg/kg body weight/day, often 0.5-1 mg/kg/day, given orally or parenterally.

Also within the scope of the disclosure is a combination therapy comprising a compound of this disclosure and one or more conventional immunosuppressive agents. These immunosuppressant agents within the scope of this disclosure include, but are not limited to, Imurek® (azathioprine sodium), brequinar sodium, Spanidin™ (gusperimus trihydrochloride, also known as deoxyspergualin), mizoribine (also known as bredinin), Cellcept® (mycophenolate mofetil), Neoral® (Cyclosporin A; also marketed as a different formulation under the trademark Sandimmune®), Prograf™ (tacrolimus, also known as FK-506), Rapimmune® (sirolimus, also known as rapamycin), leflunomide (also known as HWA-486), Zenapax®, glucocortcoids, such as prednisolone and its derivatives, antibodies such as orthoclone (OKT3), and antithymyocyte globulins, such as thymoglobulins. The compounds are useful as potentiators when administered concurrently with another immunosuppressive drug for immunosuppressive treatments as discussed above. A conventional immunosuppressant drug, such as those above, may thus be administered in an amount substantially less (e.g. 20% to 50% of the standard dose) than when the compound is administered alone. Alternatively, the disclosed formulation is administered in amounts such that the resultant immunosuppression is greater than what would be expected or obtained from the sum of the effects obtained with the drug and disclosed compound used alone. Typically, the immunosuppressive drug and potentiator are administered at regular intervals over a time period of at least 2 weeks.

The compositions of the disclosure may also be administered in combination with a conventional anti-inflammatory drug (or drugs), where the drug or amount of drug administered is, by itself, ineffective to induce the appropriate suppression or inhibition of inflammation.

Immunosuppressive activity of compounds in vivo can be evaluated by the use of established animal models known in the art. Such assays may be used to evaluate the relative effectiveness of immunosuppressive compounds and to estimate appropriate dosages for immunosuppressive treatment. These assays include, for example, a well-characterized rat model system for allografts, described by Ono and Lindsey (1969), in which a transplanted heart is attached to the abdominal great vessels of an allogeneic recipient animal, and the viability of the transplanted heart is gauged by the heart's ability to beat in the recipient animal. A xenograft model, in which the recipient animals are of a different species, is described by Wang (1991) and Murase (1993). A model for evaluating effectiveness against GVHD involves injection of normal F1 mice with parental spleen cells; the mice develop a GVHD syndrome characterized by splenomegaly and immunosuppression (Korngold, 1978; Gleichmann, 1984). Single cell suspensions are prepared from individual spleens, and microwell cultures are established in the presence and absence of concanavalin A to assess the extent of mitogenic responsiveness.

### VI. ANTICANCER TREATMENT

The following disease states have been shown to be amenable to treatment with triptolide and its prodrugs and other analogs. Such disease states are target areas for treatment with second-generation triptolide analogs. Triptolide analogs and/or prodrug compounds also may be used in combination with conventional therapeutic agents.

As used herein, “cancer” refers to all types of cancer or neoplasm or malignant tumors found in mammals especially humans, including leukemias, sarcomas, carcinomas and melanoma. Examples of cancers are cancer of the brain, breast, cervix, colon, head and neck, kidney, lung, non-small cell lung, melanoma, mesothelioma, ovary, sarcoma, stomach, uterus and medulloblastoma. The term “leukemia” refers broadly to progressive, malignant diseases of the blood-forming organs and is generally characterized by a distorted proliferation and development of leukocytes and their precursors in the blood and bone marrow. The term “sarcoma” generally refers to a tumor which is made up of a substance like the embryonic connective tissue and is generally composed of closely packed cells embedded in a fibrillar or homogeneous substance. The term “melanoma” is taken to mean a tumor arising from the melanocytic system of the skin and other organs. The term “carcinoma” refers to a malignant new growth made up of epithelial cells tending to infiltrate the surrounding tissues and give rise to metastases.

Included, for example, are cancers involving cells derived from reproductive tissue (such as Sertoli cells, germ cells, developing or more mature spermatogonia, spermatids or spermatocytes and nurse cells, germ cells and other cells of the ovary), the lymphoid or immune systems (such as Hodgkin's disease and non-Hodgkin's lymphomas), the hematopoietic system, and epithelium (such as skin, including malignant melanoma, and gastrointestinal tract), solid organs, the nervous system, e.g. glioma (see Y. X. Zhou et al., 2002), and musculoskeletal tissue. The compounds may be used for treatment of various cancers, including, but not limited to, cancers of the brain, head and neck, lung, thyroid, breast, colon, ovary, cervix, uterus, testicle, bladder, prostate, liver, kidney, pancreas, esophagus and/or stomach. Treatment of breast, colon, lung, and prostate tumors is particularly contemplated. Treatment is targeted to slowing the growth of tumors, preventing tumor growth, inducing partial regression of tumors, and inducing complete regression of tumors, to the point of complete disappearance, as well as preventing the outgrowth of metastases derived from solid tumors. Additional cancers which can be treated with compounds according to the disclosure include, for example, multiple myeloma, medulloblastoma, lymphoma, neuroblastoma, melanoma, premalignant skin lesions, rhabdomyosarcoma, primary thrombocytosis, primary macroglobulinemia, small-cell lung tumors, non-small cell lung, large cell lung, primary brain tumors, endometrial cancer, malignant pancreatic insulinoma, malignant carcinoid, malignant hypercalcemia, and adrenal cortical cancer.

The compositions may be administered to a patient afflicted with cancer and/or leukemia by any conventional route of administration, as discussed above. The method is useful to slow the growth of tumors, prevent tumor growth, induce partial regression of tumors, and induce complete regression of tumors, to the point of complete disappearance. The method is also useful in preventing the outgrowth of metastases derived from solid tumors.

The compositions of the disclosure may be administered as sole therapy or with other supportive or therapeutic treatments not designed to have anti-cancer effects in the subject. The method also includes administering the disclosure compositions in combination with one or more conventional anti-cancer drugs or biologic protein agents, where the amount of drug(s) or agent(s) is, by itself, ineffective to induce the appropriate suppression of cancer growth, in an amount effective to have the desired anti-cancer effects in the subject. Such anti-cancer drugs include actinomycin D, camptothecin, carboplatin, cisplatin, cyclophosphamide, cytosine arabinoside, daunorubicin, doxorubicin, etoposide, fludarabine, 5-fluorouracil, hydroxyurea, gemcitabine, irinotecan, methotrexate, mitomycin C, mitoxantrone, paclitaxel, taxotere, teniposide, topotecan, vinblastine, vincristine, vindesine, and vinorelbine. Anti-cancer biologic protein agents include tumor necrosis factor (TNF), TNF-related apoptosis inducing ligand (TRAIL), other TNF-related or TRAIL-related ligands and factors, interferon, interleukin-2, other interleukins, other cytokines, chemokines, and factors, antibodies to tumor-related molecules or receptors (such as anti-HER2 antibody), and agents that react with or bind to these agents (such as members of the TNF super family of receptors, other receptors, receptor antagonists, and antibodies with specificity for these agents).

Antitumor activity in vivo of a particular composition can be evaluated by the use of established animal models, as described, for example, in Fidler et al., U.S. Pat. No. 6,620,843. Clinical doses and regimens are determined in accordance with methods known to clinicians, based on factors such as severity of disease and overall condition of the patient.

A compound of structure I, 18-deoxo-19-dehydro-18-benzoyloxy-19-benzoyl triptolide (designated PG796), was cytotoxic to Jurkat cells (according to Example 2) in a dose-dependent manner. Thus, the present disclosure includes the use of the disclosed compounds as cytotoxic agents, particularly to treat cancers.

### VII. OTHER INDICATIONS

The compounds of the present disclosure may also be used in the treatment of certain CNS diseases. Glutamate fulfills numerous physiological functions, but also plays an important role in the pathophysiology of different neurological and psychiatric diseases. Glutamate excitotoxicity and neurotoxicity have been implicated in hypoxia, ischemia and trauma, as well as in chronic neurodegenerative or neurometabolic diseases, Alzheimer's dementia, Huntington's disease and Parkinson's disease. In view of the reported neuroprotective effects of triptolide, particularly protection from glutamate-induced cell death (Q. He et al., 2003; X. Wang et al., 2003), compounds of the disclosure are envisioned to antagonize the neurotoxic action of glutamates and thus may be a novel therapy for such diseases.

Recent evidence from MS patients in relapse suggests an altered glutamate homeostasis in the brain of patients with MS. Neurotoxic events occur in MS, and they can be responsible for oligodendrocyte and neuronal cell death in patients with this demyelinating disease. Antagonizing glutamate receptor-mediated excitotoxicity by treatment with compounds of this disclosure may have therapeutic implications in MS patients. Other nervous system diseases such as, Guillain-Barre syndrome, Meniere's disease, polyneuritis, multiple neuritis, mononeuritis and radiculopathy may be treated with the compounds of the present disclosure.

The compounds of the present disclosure may also be used in the treatment of organ fibrosis, including certain lung diseases. Idiopathic pulmonary fibrosis (PF) is a progressive interstitial lung disease with no known etiology. PF is characterized by excessive deposition of intracellular matrix and collagen in the lung interstitium and gradual replacement of the alveoli by scar tissue as a result of inflammation and fibrosis. As the disease progresses, the increase in scar tissue interferes with the ability to transfer oxygen from the lungs to the bloodstream. A 14-succinimide ester of triptolide has been reported to block bleomycin-induced PF (G. Krishna et al., 2001). Accordingly, the compounds and formulations of the present disclosure may be useful for treatment of PF. Treatment of other respiratory diseases, such as sarcoidosis, fibroid lung, and idiopathic interstitial pneumonia is also considered.

Other diseases involving the lung and envisioned to be treatable by compounds of this disclosure include Severe Acute Respiratory Syndrome (SARS) and acute respiratory distress syndrome (ARDS). In particular, with respect to SARS, the reduction of virus content (SARS-CoV) before the peak of the disease process and the usefulness of corticosteroid treatment, as noted below, suggest that the development of the most severe, life-threatening effects of SARS may result from the exaggerated response of the body to the infection (immune hyperactivity) rather than effects of the virus itself (See also copending and co-owned U.S. provisional application Ser. No. 60/483,335, incorporated herein by reference.) Corticosteroid treatment has been used in SARS patients to suppress the massive release of cytokines that may characterize the immune hyperactive phase, in the hope that it will stop the progression of pulmonary disease in the next phase. Corticosteroid treatment has produced good clinical results in reduction of some of the major symptoms of SARS. However, there are several treatment-related side effects, and there is a clear need for more selective immunosuppressive and/or antiinflammatory agents.

Triptolide-related compounds may also be used in the treatment of certain CNS diseases. Glutamate fulfills numerous physiological functions, including an important role in the pathophysiology of various neurological and psychiatric diseases. Glutamate excitotoxicity and neurotoxicity have been implicated in hypoxia, ischemia and trauma, as well as in chronic neurodegenerative or neurometabolic diseases, Alzheimer's disease (AD), Huntington's disease and Parkinson's disease. In view of the reported neuroprotective effects of triptolide, particularly protection from glutamate-induced cell death (He et al., 2003; Wang et al., 2002a), compounds of the disclosure are envisioned to antagonize the neurotoxic action of glutamates and thus may be a novel therapy for such diseases.

Cerebral amyloid angiopathy is one of the pathological features of AD, and PC12 cells are extremely sensitive to induction of neurotoxicity by mutant β-amyloid protein aggregates. PC12 cells treated with β-amyloid exhibit increased accumulation of intracellular ROS and undergo apoptotic death (Gu et al., 2004). Beta-amyloid treatment induces NF-κB activation in PC12 cells, and increases the intracellular Ca²⁺ level. Triptolide has been shown to markedly inhibit β-amyloid-induced apoptosis to inhibit the increase of intracellular Ca²⁺ concentration induced by β-amyloid. Accordingly, triptolide-related compounds may be effective to prevent the apoptosis cascade induced by β-amyloid and preserve neuronal survival in AD patients.

Triptolide exerts a powerful inhibitory influence over lipopolysaccharide (LPS)-activated microglial activity by reducing nitrite accumulation, TNF-α and IL-1β release, and induction of mRNA expression of these inflammatory factors (Zhou et al., 2003). Triptolide also attenuates the LPS-induced decrease in ³H-dopamine uptake and loss of tyrosine hydroxylase-positive neurons in primary mesencephalic neuron/glia mixed culture (Li et al., 2004). Triptolide appeared to exert a neurotrophic effect without LPS. Triptolide also blocked LPS-induced activation of microglia and excessive production of TNF-α and nitrite. Triptolide may protect dopaminergic neurons from LPS-induced injury by inhibiting microglia activation, which is relevant to Parkinson's disease, further illustrating the neuroprotective potential of triptolide-related compounds.

Tripchlorolide, which has been shown to be a prodrug of triptolide, promotes dopaminergic neuron axonal elongation in primary cultured rat mesencephalic neurons and protects dopaminergic neurons from a neurotoxic lesion induced by 1-methyl-4-phenylpyridinium ion (Li et al., 2003). Tripchlorolide stimulates brain-derived neurotrophic factor mRNA expression as revealed by in situ hybridization. Furthermore, in an in vivo rat model of PD in which FK506 shows neurotrophic activity, administration of tripchlorolide at 0.5-1 μg/kg improves recovery of rats undergoing neurosurgery, produces significant sparing of SN neurons and preservation of the dendritic processes surrounding tyrosine hydroxylase positive neurons, attenuates dopamine depletion, increases the survival of dopaminergic neurons and attenuates the elevation of TNF-α and IL-2 levels in the brain (Cheng et al., 2002). Moreover, tripchlorolide demonstrates neurotrophic activity at a concentration lower than needed for neuroprotective and immunosuppressive activity.

Recent evidence from MS patients in relapse suggests an altered glutamate homeostasis in the brain. Neurotoxic events occurring in MS patients can be responsible for oligodendrocyte and neuronal cell death. Antagonizing glutamate receptor-mediated excitotoxicity by treatment with triptolide-related compounds may have therapeutic implications in MS patients. Other CNS diseases such as Guillain-Barre syndrome, Meniere's disease, polyneuritis, multiple neuritis, mononeuritis and radiculopathy may also be treated with triptolide-related compounds.

### VIII. ACTIVE FORMULATIONS

The active ingredient can be PG796, PG763, PG762 or PG695, related structures, or any triptolide derivative with a clogP of greater than 0.5 (See Table 3, below).

The chemical structures of exemplary triptolide analogs are shown below:

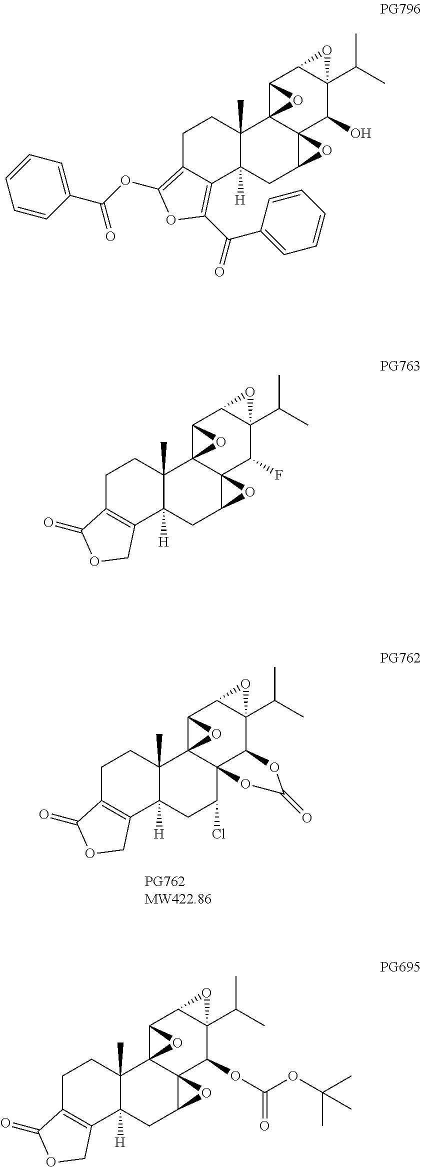 [SMILES: CC(C)[C@]12O[C@H]1[C@@H]1O[C@]13[C@]1(O[C@H]1C[C@H]1c4c(C(=O)c5ccccc5)oc(OC(=O)c5ccccc5)c4CC[C@@]13C)[C@@H]2O ; CC(C)[C@]12O[C@H]1[C@@H]1O[C@]13[C@]1(O[C@H]1C[C@H]1C4=C(CC[C@@]13C)C(=O)OC4)[C@H]2F ; CC(C)[C@]12O[C@H]1[C@@H]1O[C@]13[C@@]1(OC(=O)O[C@H]21)[C@H](Cl)C[C@H]1C2=C(CC[C@@]13C)C(=O)OC2 ; CC(C)[C@]12O[C@H]1[C@@H]1O[C@]13[C@]1(O[C@H]1C[C@H]1C4=C(CC[C@@]13C)C(=O)OC4)[C@@H]2OC(=O)OC(C)(C)C]

As is known to skilled artisans in the chemical and pharmaceutical sciences, a partition-coefficient or distribution-coefficient is the ratio of concentrations of a compound in a mixture of two immiscible phases at equilibrium. These coefficients are a measure of the difference in solubility of the compound in these two phases. Typically, one of the solvents in the mixture is water while the second is hydrophobic such as octanol. Thus, the partition-coefficient is a measure of how hydrophilic (“water-loving”) or hydrophobic (“water-fearing”) a chemical substance is. In medical practice, partition coefficients are useful for example in estimating distribution of drugs within the body. Hydrophobic drugs with high octanol/water partition coefficients are preferentially distributed to hydrophobic compartments such as lipid bilayers of cells while hydrophilic drugs (low octanol/water partition coefficients) preferentially are found in hydrophilic compartments such as blood serum. Thus, a formulation can be characterized by its solubility in both water and fat, as an orally administered drug needs to pass through the intestinal lining after it is consumed, carried in aqueous blood and penetrate the lipid cellular membrane to reach the inside of a cell. A model compound for the lipophilic cellular membrane is octanol (a lipophilic hydrocarbon), so the logarithm of the octanol/water partition coefficient, known as “LogP,” is used to predict the solubility of a potential oral drug. This coefficient can be experimentally measured or predicted computationally, in which case it is sometimes called a “calculated partition coefficient” or “cLogP.”

TABLE 3
cLogP of triptolide and triptolide analogs/derivatives

| | | cLogP | |
|---|---|---|---|
| Compound | Chemical Class | Method A | Method B |
| triptolide | | −0.08 | 0.27 |
| PG796 (MRx102) | lactone | 3.68 | 4.11 |
| PG763 (MRx103) | halogens | 0.63 | 0.87 |
| PG762 (MRx104) | c-ring | 1.60 | 1.89 |
| PG490-88 (MRx108) | esters | −0.18 | 0.19 |
| PG695 (MRx109) | carbonates | 1.61 | 1.85 |

Method A - Crippen's fragmentation: J. Chem. Inf. Comput. Sci., 27, 21(1987)
Method B - Viswanadhan's fragmentation: J. Chem. Inf. Comput. Sci., 29, 163(1989)

From a survey of the literature, it is possible to obtain some general guidelines about the optimum Log P values for certain classes of drugs. (See A guide to Log P and pKa measurements and their use by Mark Earll www.raell.demon.co.uk/chem/logp/logppka.html). In general, assuming passive absorption,

Optimum CNS penetration approximately Log P=2+/−0.7 (Hansch)

Optimum Oral absorption approximately Log P=1.8

Optimum Intestinal absorption Log P=1.35

Optimum Colonic absorption LogP=1.32

Optimum Sublingual absorption Log P=5.5

Optimum Percutaneous Log P=2.6 (& low mw)

Formulation and dosing forms:

Low Log P (below 0) Injectable

Medium (0-3) Oral

High (3-4) Transdermal

Very High (4-7) Toxic build up in fatty tissues

Overall, triptolide compounds having a cLogP of 0.5 or higher are believed not to be amenable to formulations meant for injection. For example, of the compounds in Table 3, compounds PG796, PG763, PG762 or PG695 were generally predicted by skilled artisans to not have a workable cLogP for injectable intravenous administration. Unexpectedly, however, an effective injectable formulation for compounds having a cLogP of 0.5 or higher (such as, for example, PG796, PG763, PG762 or PG695) has been designed and is identified hereinbelow.

### EXAMPLES

The following examples are illustrative in nature and are in no way intended to be limiting.

### Example 1

### Emulsion Preparation

Emulsion components include glyceryl trioctanoate (g) 20%; Soybean oil (g) 20%; Phospholipids ([60%] L-α-phosphatidylcholine, L-lecithin, Sigma 61755) (g) 2%; Sodium cholate (g) 0.2%; Glycerin (g) 2.5%; Water (ml) 55%

Emulsion Preparation with PG796(MRx102)
1. Weigh glyceryl trioctanoate, soybean oil, and phospholipids (L-lecithin) into a 15 mL conical plastic centrifuge tube or a suitable test tube (e.g., plastic to avoid breakage).
2. Place the tube over the bottom of the sonicator probe such that the sonicator tip is about 5 mm from the bottom of the tube and the probe is not in contact with the sides of the tube. Clamp it in place. Do not use a cold water bath at this stage.
3. Set the sonicator to a power level a little below the microtip limit and at a duty cycle of 50%. Turn the sonicator on for 20 seconds.
4. Feel the tube to assess its temperature and observe the contents carefully to determine whether the phospholipids are dispersing. Sonicators are very efficient at generating shear energy and cavitation, but are not efficient mixers, so it might be necessary to unclamp the tube and use the probe as a stirrer to break up the phospholipids.
5. In order to disperse the phospholipids, the fluid should be allowed to warm to 40° C.-50° C. Continue sonicating for short intervals until the fluid is warm, but not hot to the touch. Once the fluid has warmed up, suspend the tube in a beaker of warm water and continue sonicating for five minutes or until full dispersion of the phospholipids has been obtained, whichever is longer.
6. Weigh and add PG796(MRx102) in the fluid that is about 20° C.-25° C. Sonicate the solution for short intervals (each about 20 seconds) until the dissolution of the PG796(MRx102) has been obtained. After each interval sonicating, suspend the tube in a beaker of water (about 15° C.-20° C.) to cool down the temperature to make sure the temperature is lower than to 40-45° C. It may take about 10 interval sonicatings to dissolve PG796(MRx102) completely.
7. Measure/weigh the water and sodium cholate into a beaker and dissolve the sodium cholate. Add and dissolve the glycerin into the sodium cholate solution.
8. Suspend the phospholipid/oil/PG796(MRx102) tube in a cold water bath and add about ⅓ of the water/sodium cholate/glycerin mixture, sonicate for 1 minute with the tube in the cold water bath by adjusting the sonicator to a power level a little below the microtip limit (about 4.9).
9. Add the second third of the water/sodium cholate/glycerin mixture and repeat sonication for 1 minute. Add the last of the water/sodium cholate/glycerin mixture and sonicate for another 1 minute. Sonicate further if the water/sodium cholate/glycerin mixture is not completely dissolved in the emulsion.
10. Remove the tube from the sonication probe and check the pH (around 7.6 for this formulation). Carefully adjust the pH to be in the range of 7.5 to 8.5 using 0.1N sodium hydroxide if necessary. A pH closer to 7.5 is suitable physiologically for dosing animals.
11. Place the tube back on the sonication probe in the cold water bath and sonicate for 8 minutes continuously.
12. Note that the emulsion should be opaque white, similar to thick cream.
13. Filter the emulsion through a 0.45 μm membrane filter (Polyethersulfone 0.45 μm Pore Size filter, such as Millipore Millex-HP Syringe Filter Unit SLHPM33RS, Radio-Sterilized). The emulsion preferably appears unchanged.
14. Introduce the emulsion containing PG796(MRx102) into test subject for appropriate studies.

Components for Preparation of 5 ml of Emulsion with PG796(MRx102)

| Components with PG796(MRx102) | Amount |
|---|---|
| Glyceryl trioctanoate (g) | 1 |
| Soybean oil (g) | 1 |
| Phospholipids (g) | 0.1 |
| Glycerin (g) | 0.125 |
| Sodium cholate (g) | 0.01 |
| PG796(MRx102) (mg) | 5 |
| Water (ml) | 2.77 |

Component (Excipient) Range

Formulation

| Components | Range | E-0212-4 |
|---|---|---|
| Glyceryl trioctanoate | 0%-50% | 20% |
| Soybean oil | 0%-45% | 20% |
| Phospholipids | 1%-3% | 2% |
| Glycerin | 1%-5% | 3% |
| Sodium cholate | 0.1%-0.3% | 0.2% |
| Water | 50%-60% | 55% |

Alternative Components (Excipients)

Alternative components or excipients are indicated below.

1. Glyceryl trioctanoate include

a. glyceryl trihexanoate

b. glyceryl triheptanoate,

c. glyceryl trinonanoate,

d. glyceryl tridecanoate

2. Soybean oil

a. castor oil,

b. corn oil,

c. cottonseed oil,

d. olive oil,

e. peanut oil,

f. peppermint oil,

g. safflower oil,

h. sesame oil,

i. hydrogenated vegetable oils,

j. hydrogenated soybean oil, and

k. medium-chain triglycerides of coconut oil

l. medium-chain triglycerides palm seed oil

3. Phospholipids

a. hydrogenated soy phosphatidylcholine,

b. distearoylphosphatidylglycerol,

c. L-alpha-dimyristoylphosphatidylcholine,

d. L-alpha-dimyristoylphosphatidylglycerol

4. Glycerin

a. polyethylene glycol 300,

b. polyethylene glycol 400,

c. ethanol,

d. propylene glycol,

e. N-methyl-2-pyrrolidone,

f. dimethylacetamide, and

g. dimethylsulfoxide

5. Sodium cholate

a. sodium taurocholate,

b. sodium tauro-β-muricholate,

c. sodium taurodeoxycholate,

d. sodium taurochenodeoxycholate,

e. sodium glycocholate,

f. sodium glycodeoxycholate and

g. sodium glycochenodeoxycholate

Alternatively, the protocol above may be performed through the first part of step 8, above, whereby PG796(MRx102) is suspended/dissolved in the phospholipid/oil mixture, and the suspension/solution can then be stored as a drug product. Accordingly, the composition is anhydrous, minimizing the potential for hydrolysis of the triptolide or triptolide analog, the shelf life can be prolonged, and the water/sodium cholate/glycerin mixture can then be added according to step 8 and the remainder of the protocol can be carried out, continuing through step 14 above, at the time of administration to a subject.

Similarly, to aid in stability, dispersion and filtration, the composition can be sterilized (e.g., filtration, autoclaving), and/or other excipients may be added to favor globules of a desired size.

Preliminary Emulsion Evaluation

Pharmaceutical emulsions intended for administration by injection or infusion typically consist of a triglyceride such as soybean oil (SBO) with naturally derived phospholipids (egg yolk or soy) emulsified with use of a high pressure homogenizer. Nonionic surfactants such as Tweens (polysorbates), Solutol®, and Kolliphor (Cremophor®), are generally not used in formulations for injection or infusion, because they undergo phase inversions with heating, and injectable emulsions are usually heat sterilized. Nonetheless, some preliminary investigations were initiated with nonionic surfactants.

Various ratios of the nonionic surfactants polysorbate 80 (a.k.a. Tween 80) and Span 80 were explored, and a formulation was prepared and tested as follows. Glyceryl trioctanoate (GTO) was used as the triglyceride oil, as PG796(MRx102) had been shown to be about 3.4 fold more soluble in GTO than in SBO. The formulation and results are shown in Table 4. The results of this preliminary experiment were encouraging in that a reasonably high solubility was obtained in a formulation containing almost 70% water.

TABLE 4
Preliminary emulsion formulation and solubility.

| | | | | PG796(MRx102) |
|---|---|---|---|---|
| GTO | Span 80 | Tween 80 | water | Solubility (μg/ml) |
| 29.4% | 1.65% | 0.31% | 68.6% | 681 |

Due to the lack of availability of a co-solvent/surfactant formulation with an acceptable side effect profile when injected intravenously into rats, emulsions were considered. The following characteristics were selected as desirable for an emulsion formulation:

As a vehicle alone, poses no overt side effects in vivo (rodents),

Has >2 mg/ml PG796(MRx102) stable concentration,

Retains 95% PG796(MRx102) concentration after filtration,

Possesses 7 days of acceptable stability, and

Is compatible with MRx100.

Emulsion formulations were prepared using a probe sonicator to disperse the oil phase in the aqueous phase to form a creamy opaque suspension.

Range-Finding Formulations

Typical emulsion formulations consist of 10-30% triglyceride, most commonly SBO, dispersed with 0.5-2% phospholipids in an aqueous phase, which contains glycerin as a tonicity agent. However because of the low solubility observed for PG796(MRx102), initial formulations were prepared with 40% of GTO, a medium chain triglyceride in which PG796(MRx102) was found to have higher solubility. Additionally, PEG-400 and ethanol were incorporated into some of the formulations to decrease the polarity of the aqueous phase to enhance solubility. Sodium cholate was included in some formulations as a co-surfactant. The formulations, along with visual assessments and PG796(MRx102) solubility values are shown in Table 5. Solubility of at least 1 mg/mL was obtained in all of the formulations. In each case some loss of potency was observed after eight days of storage, but the majority of the original potency was maintained. PEG-400 and ethanol were only marginally beneficial in improving solubility, and one of the formulations containing PEG-400 failed to form a homogenous emulsion.

TABLE 5
First round emulsion formulations and solubilities

| | | Formulation # | | | | |
|---|---|---|---|---|---|---|
| Components | | E-1 | E-2 | E-3 | E-4 | E-5 |
| Glyceryl trioctanoate | | 40% | 40% | 40% | 40% | 40% |
| Phospholipids | | 2% | 2% | 2% | 2% | 2% |
| PEG-400 | | 10% | — | — | 10% | — |
| Ethanol | | — | 10% | — | — | 10% |
| Sodium cholate | | — | — | 0.2% | 0.2% | 0.2% |
| Water | | 48% | 48% | 58% | 48% | 48% |
| PG796 (MRx102) | | 2 mg/mL | | | | |
| Visual assessment | | 2 | homog- | homog- | homog- | homog- |
| | | layers | enous | enous | enous | enous |
| PG796 | 0 hr | 1560 | 1913 | 1529 | 1787 | 1673 |
| (MRx102) | 1 hr | 1677 | 1879 | 1514 | 1795 | 1680 |
| Solubility | 24 hr | 1484 | 1939 | 1353 | 1762 | 1654 |
| (μg/mL) | 8 days | — | 1353 | 1176 | 1470 | 1329 |

Effect of pH on Stability

Pharmaceutical emulsions are typically prepared at neutral to slightly alkaline pH since they are stabilized by electrostatic repulsion between droplets imparted by pH-sensitive anionic surfactants, such as phosphatidyl ethanolamine, free fatty acid salts, and cholate. However, it was possible that this pH range could be suboptimal for the chemical stability of PG796(MRx102). To test this, emulsions were prepared at different pH values ranging from 4 to 8. Buffers were included to control the pH, and the non-pH sensitive surfactant, sodium dodecyl sulfate was used in place of sodium cholate to assure a negative charge even in the low pH emulsions. Formulations and results are shown in Table 6. All of the formulations were reasonably stable for up to 2 weeks at room temperature. Although there was some variation in potency and purity, there was no trend with pH, indicating that the stability of PG796(MRx102) in the emulsion is not pH-dependent within this range.

TABLE 6
Effect of pH on stability of PG796 (MRx102) in emulsions.

| | | Target pH | | | | |
|---|---|---|---|---|---|---|
| Components | | 4.0 | 5.0 | 6.0 | 7.0 | 8.0 |
| Glyceryl trioctanoate | | 40% | 40% | 40% | 40% | 40% |
| Phospholipids | | 2% | 2% | 2% | 2% | 2% |
| Ethanol | | 10% | 10% | 10% | 10% | 10% |
| 0.1% SDS in buffer | | 48% | 48% | 48% | 48% | 48% |
| Buffer (10 mM) | | acetate | acetate | histi- | phos- | Tris |
| | | | | dine | phate | |
| PG796 (MRx102) | | 1 mg/mL | | | | |
| Measured pH | | 4.06 | 4.97 | 5.98 | 7.03 | 8.05 |
| PG796 | 0 hr | 870 | 1006 | 1093 | 996 | 929 |
| (MRx102) | 24 hr | 917 | 922 | 890 | 849 | 929 |
| Solubility | 1 wk | 1001 | 972 | 948 | 760 | 1041 |
| (μg/mL) | 2 wk | 848 | 910 | 850 | 822 | 930 |
| PG796 | 0 hr | 98.9 | 99.2 | 99.2 | 99.4 | 98.7 |
| (MRx102) | 24 hr | 99.3 | 99.4 | 99.2 | 99.1 | 99.2 |
| Purity | 1 wk | 98.9 | 99.1 | 98.9 | 98.8 | 99.1 |
| peak area % | 2 wk | 97.2 | 98.3 | 98.8 | 91.0 | 98.3 |

Second Round Emulsion Formulations

To modify the 40% glyceryl trioctanoate vehicles, formulations were prepared using a lower level of triglyceride and/or partial or complete substitution of soybean oil for glyceryl trioctanoate. These formulations and solubility data obtained with them are shown in Table 7. When two values are listed, these are for duplicate analyses. The formulations were heat sterilized for 8 minutes at 121° C. A placebo version of formulation E-0212-4 was also prepared and sterilized to determine the level of placebo component co-elution in HPLC analysis, and this was found to be 1.23%.

As expected, reducing the triglyceride content and replacing some or all of the glyceryl trioctanoate with soybean oil led to some drop in drug solubility. However, only in formulation E-0212-1, in which the triglyceride content was dropped from 40% to 30% and all of the GTO was replaced with soybean oil, was PG796(MRx102) solubility much less than 1 mg/mL.

TABLE 7
Second Round Emulsion Formulations

| | | Formulation # | | | | |
|---|---|---|---|---|---|---|
| Components | | E-0212-1 | E-0212-2 | E-0212-3 | E-0212-4 | E-0212-5 |
| Glyceryl trioctanoate | | — | 15% | 30% | 20% | — |
| Soybean oil | | 30% | 15% | — | 20% | 40% |
| Phospholipids | | 2% | 2% | 2% | 2% | 2% |
| Glycerin | | 3% | 3% | 3% | 3% | 3% |
| Sodium cholate | | 0.2% | 0.2% | 0.2% | 0.2% | 0.2% |
| Water | | 65% | 65% | 65% | 55% | 55% |
| PG796 (MRx102) | | 1 mg/mL | | | | |
| PG796 | initial | 682 | 929, 928 | 968 | 1090, 991 | 934, 867 |
| (MRx102) | sterilized | 621 | 771, 847 | 913 | 1046, 905 | 913, 867 |
| Solubility | | | | | | |
| (μg/mL) | | | | | | |
| PG796 | initial | 96.2% | 96.5, 96.6% | 97.9% | 98.1, 97.3% | 95.5, 96.1% |
| (MRx102) | sterilized | 94.6% | 95.6, 96.3% | 97.2% | 97.4, 96.7% | 99.6, 95.9% |
| Purity | | | | | | |
| (peak area %) | | | | | | |

Toxicological Observations with Emulsions

Rats were administered an intravenous bolus of 5 mL/kg of formulation E-3 (40% GTO, 2% phospholipids, 0.2% sodium cholate). The animals appeared normal immediately after injection but became lethargic and were then recumbent with labored breathing within 5-10 minutes. The rats recovered and appeared to be normal within 60-90 minutes. A second dose administered the following day appeared to cause more severe symptoms. Injections given the next 2 days produced similar responses. A second cohort of rats was administered an intravenous bolus of 5 mL/kg of formulation E-5 (the same formulation as E-3 but with addition of 10% ethanol). All of the animals were recumbent and immobile after 10 minutes and died after about 45 minutes.

Formulation E-3 was tested at the higher concentration of 2 mg/mL PG796(MRx102), which was found to be soluble. The higher concentration would allow dosing at a commensurately lower volume. Accordingly, a cohort of rats was administered a reduced dose of 1.5 mL/kg of formulation E-3. The animals appeared normal for 8-10 minutes after injection, and were then recumbent for 8-10 minutes. Thus the adverse events were less severe, and the period of recumbency and the recovery times were shorter with this dose. The three experiments are summarized in Table 8.

TABLE 8
Initial rat studies with emulsion formulations.

| | Experiment 1 | | Experiment 2 | Experiment 3 | |
|---|---|---|---|---|---|
| | Formulation | | | | |
| Components | E-3 | | E-5 | E-3 | |
| Glyceryl trioctanoate | 40% | | 40% | 40% | |
| Phospholipids | 2% | | 2% | 2% | |
| Ethanol | — | | 10% | — | |
| Sodium cholate | 0.2% | | 0.2% | 0.2% | |
| Water | 58% | | 48% | 58% | |
| Volume injected i.v. | 5 | ml/kg | 5 ml/kg | 1.5 | ml/kg |
| Deaths | 0 of 4 | | 4 of 4 | 0 of 4 | |
| Recovery Time | 60-90 | min | N/A | 15-17 | min |

In a comparison of emulsions with only soybean oil (40%, emulsion E0212-4) and an equal mixture of glyceryl trioctanoate and soybean oil (20% of each, emulsion E0212-5), rats were administered an intravenous bolus of 3 mL/kg daily for 4 days. On the first day of injection, the animals that were given E0212-4 became slightly lethargic at 7 min, and were fully recovered by 40 min. The E0212-5 rats were slightly lethargic at 8 min, and they had recovered fully by 35 min. Previous tests had shown rats to be recumbent for a protracted period after the intravenous injection of various emulsion formulations, more severe symptoms. The result is improved with these two newest emulsion formulations when injected intravenous into rats. The side effects for the emulsion injections given to rats on days 2-4 were very similar to those observed on Day 1. The use of 40% SBO did not completely eliminate side effects seen with 20% GTO/20% SBO. Side effects observed after the first injection were less severe than those of Formulation #3 at 5 ml/kg and even at 1.5 ml/kg. There was no labored breathing, and there was only slight lethargy in contrast to the earlier studies showing labored breathing and lethargy.

The 20% GT/20% SBO emulsion formulation (E-0212-4) showed an acceptable chemical solubility/stability profile, was non-lethal in tests of the vehicle alone in rat studies, and caused minimal side effects (less than other emulsion formulation preparations), it was selected as the revised vehicle formulation for use in the Escalating Dose/7-Day Repeat Dose Comparison Study of PG796(MRx102) and MRx100 in rats, and the Escalating Dose/7-Day Repeat Dose Study of PG796(MRx102) in dogs.

TABLE 9
Side effect rat studies comparing emulsion formulations

| | Formulation | | |
|---|---|---|---|
| Components | | E-0212-4 | E-0212-5 |

| | | |
|---|---|---|
| Glyceryl trioctanoate | 20% | — |
| Soybean oil | 20% | 40% |
| Phospholipids | 2% | 2% |
| Glycerin | 3% | 3% |
| Sodium cholate | 0.2% | 0.2% |
| Water | 55% | 55% |

| | | | | |
|---|---|---|---|---|
| Volume injected i.v. | 3 | ml/kg | 3 | ml/kg |

| | | |
|---|---|---|
| Deaths | 0 of 4 | 0 of 4 |

| | | | | |
|---|---|---|---|---|
| Recovery Time | 40 | min | 35 | min |

Pharmacokinetic/Toxicokinetic Considerations

Triptolide's molecular mechanism of action has remained elusive, but triptolide was reported to covalently bind to human XPB (also known as ERCC3), a subunit of the transcription factor TFIIH, and to inhibit its DNA-dependent ATPase activity, leading to inhibition of RNA polymerase II-mediated transcription and likely nucleotide excision repair. The identification of XPB as the target of triptolide accounts for the many of the known biological activities of triptolide. For example, triptolide binding to XPB lead to the down regulation of a number of growth and survival promoters including NF kappa B (NF-κB) and the anti-apoptotic factors Mcl-1 and XIAP. (Titov, et al., Nat. Chem. Biol. (2011) 7(3):182-8). Subsequently, the triptolide derivative MRx102 was also found to have these effects, i.e., reduced mRNA levels, reduced NF-κB and reduced Mcl-1 and XIAP. At low nanomolar concentrations, MRx102 also induced apoptosis in bulk, CD34(+) progenitor, and more importantly, CD34(+)CD38(−) stem/progenitor cells from AML patients, even when they were protected by coculture with bone marrow derived mesenchymal stromal cells. In vivo, MRx102 greatly decreased leukemia burden and increased survival time in non-obese diabetic/severe combined immunodeficiency mice harboring Ba/F3-ITD cells. Thus, MRx102 has potent antileukemic activity both in vitro and in vivo, has the potential to eliminate AML stem/progenitor cells and overcome microenvironmental protection of leukemic cells, and warrants clinical investigation. (Carter, et al., (2012) Leukemia 26:443-50). Furthermore, triptolide and triptolide derivatives can serve as a new molecular probe for studying transcription and, potentially, as a new type of anticancer agent through inhibition of the ATPase activity of XPB.

Another consequence of XPB binding is the inhibition of nucleotide excision repair. This activity in blocking DNA repair should enhance the activities of those drugs that have DNA as their target, including cisplatin and topoisomerase 1 inhibitors for solid tumors; both have been shown to act in a synergistic fashion with triptolide. The potential synergy between MRx102 and two drugs used in AML, cytarabine and idarubicin was investigated using MV4-11 cells in vitro and synergy was demonstrated between MRx102 and both of these drugs used in AML.

One concern regarding triptolide and triptolide derivatives is their epoxide structure, viewed as potentially toxic; however, proteosome inhibitor anti-cancer drug, carfilzomib (Kyprolis) is a tetrapeptide epoxyketone containing an epoxide, and was recently FDA approved. Furthermore, triptolide, even though it is a triepoxide, was shown by Titov, et al., (supra) to be exquisitely selective, and not promiscuous, in its binding characteristics. Nonetheless, triptolide's reported safety issues in a number of animal studies as well as clinically, have resulted in an “image problem” and potential safety challenges; accordingly, triptolide has not been deemed appropriate for clinical use and has not been commercially developed.

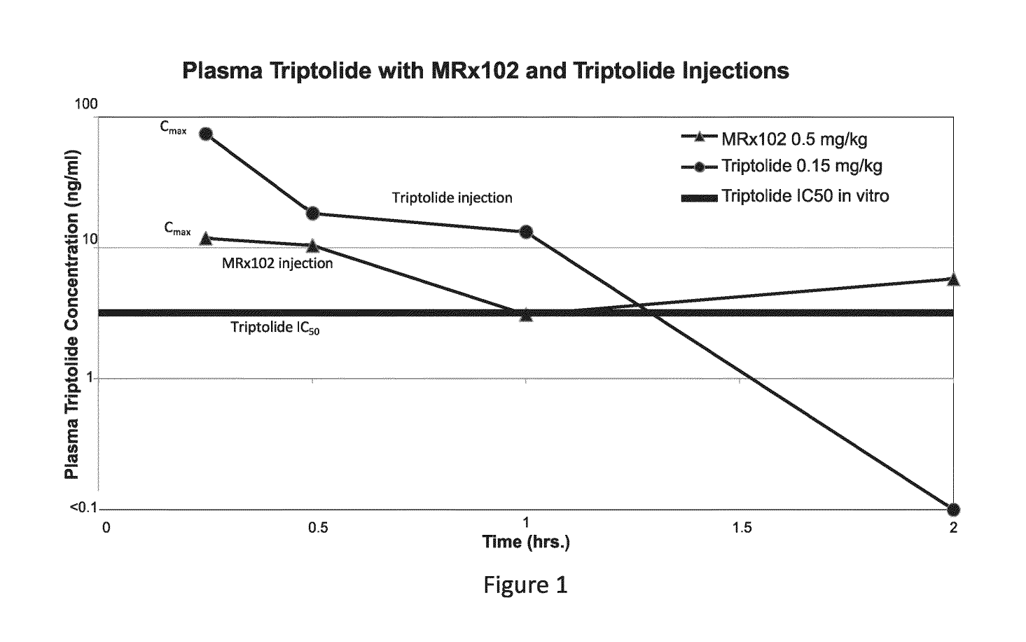

Triptolide prodrugs are generally believed to be safer than triptolide. In an initial rodent toxicology study, PG796(MRx102) demonstrated no gross or histopathologic toxic effects at intravenous doses up to 1.5 mg/kg/day for seven days. Triptolide prodrugs as an emulsion formulation are believed to have a toxicokinetic profile characterized by a flat AUC with a minimized Cmax. [In conjunction, it was postulated that a sustained inhibition of RNA polymerase is needed for optimum efficacy which in turn requires a pharmcokinetic profile of constant exposure to drug]. FIG. 1 shows a side-by-side comparative toxicology study of PG796(MRx102) and triptolide in which both drugs were administered intravenously to rodents using the novel emulsion formulation disclosed herein demonstrated that PG796(MRx102) was at least 20 times less toxic than triptolide based on both gross and histopathologic criteria. The no effect dose (“NOAEL”) of PG796(MRx102) again exceeded 1.5 mg/kg/day intravenously for seven days in rodents confirming the initial results. It is interesting to ask why a prodrug of triptolide would be safer than the natural product itself; while not wishing to be bound by theory, perhaps the answer lies in the pharmacokinetic profile of triptolide administered either directly or released from its carrier, PG796(MRx102). When triptolide is provided alone (see line connecting circles in FIG. 1) it had a very high Cmax as well as a rapid decline such that by two hours post-dose none remained in circulation. However, when the prodrug PG796(MRx102) was administered, the triptolide Cmax was approximately one-tenth that noted when triptolide was administered directly (see line connecting triangles in FIG. 1) and the triptolide blood levels remained relatively constant and demonstrate a longer AUC (“area under the curve”) as seen at the two-hour time point. It also remained above the therapeutic levels (shown as a thick line without symbols). The difference in the Cmax/AUC profile of PG796(MRx102) vs. triptolide is believed to be due to the physiochemical properties of the lipid prodrug/emulsion formulation combination. In general, triptolide prodrugs having a cLogP greater than 0.5 are more lipid-soluble than water soluble and are expected to take longer to convert to the drug form; such characteristics may yield a flatter conversion profile and less of a drug-release Cmax spike.

PG490-88 given intravenously, entered clinical trials and showed promising activity in patients with AML. (Xia Zhi Lin and Zhen You Lan, Haematologica, 93:14 (2008)). However, as a prodrug, it was incompletely and erratically converted to the active entity, triptolide, and, as such, may provide a reason it produced toxicity. However, PG490-88 did have an optimized AUC, relatively flat over time with no intense Cmax. The finding that PG796(MRx102) was rapidly and completely converted to triptolide using human serum (as well as seen in vivo in rats and dogs) while PG490-88Na was incompletely converted to triptolide in human serum argues that the conversion of PG796(MRx102) is not dependent on variations in species enzymatic (esterase) activities but is dependent on the physiochemical properties of the lipid prodrug/emulsion formulation.

Lipid emulsions have been studied as drug delivery systems for some time. (See Hippalgaonkar, et al., (2010) AAPS Pharm. Sci. Tech. 11(4):1526-1540; Stevens, et al., (2003) Business Briefing: Pharmatech 2003, p. 1-4). Solid lipid nanoparticle (SLN) delivery systems may have advantages over conventional formulations of bioactive plant extracts, such as enhancing solubility and bioavailability, offering protection from toxicity, and enhancing pharmacological activity. A tripterygium glycoside (TG) solid lipid nanoparticle (TG-SLN) delivery system was reported to have a protective effect against TG-induced male reproductive toxicity. Triptolide (TP) was used as a model drug in a comparative study of the toxicokinetic and tissue distribution of TP-SLN and free TP in rats. A fast and sensitive HPLC-APCI-MS/MS method was developed for the determination of triptolide in rat plasma. Fourteen rats were divided randomly into two groups of 7 rats each for toxicokinetic analysis, with one group receiving free TP (450 μg/kg) and the other receiving the TP-SLN formulation (450 μg/kg). Blood was obtained before dosing and 0.083, 0.17, 0.25, 0.33, 0.5, 0.75, 1, 1.5, 2, 3 and 4 h after drug administration. Thirty-six rats were divided randomly into six equal groups for a tissue-distribution study. Half of the rats received intragastric administration of TP (450 μg/kg) and the other half received TP-SLN (450 μg/kg). At 15, 45, and 90 min after dosing, samples of blood, liver, kidney, spleen, lung, and testicular tissue were taken. TP concentration in the samples was determined by LC-APCI-MS-MS. The toxicokinetic results for the nanoformulation showed a significant increase the area under the curve (AUC) (P<0.05), significantly longer T(max) and mean retention times (MRTs) (0-t) (P<0.05), significantly decreased C(max) (P<0.05). The nanoformulation promoted absorption with a slow release character, indicating that toxicokinetic changes may be the most important mechanism for the enhanced efficacy of nanoformulations. Tissue-distribution results suggest a tendency for TP concentrations in the lung and spleen to increase, while TP concentrations in plasma, liver, kidney, and testes tended to decrease in the TP-SLN group. At multiple time points, testicular tissue TP concentrations were lower in the TP-SLN group than in free TP group. This provides an important clue for the decreased reproductive toxicity observed with TP-SLN. Overall, an orally administered lipid nanoparticle formulation of triptolide promoted absorption with a slow release character. (Xue, et al., (2012) Eur. J. Pharm. Sci., 47(4):713-7). The toxicokinetic results for the nanoformulation showed a significant increase in AUC, and a decreased Cmax. These results indicate that toxicokinetic change are a consideration for enhanced safety.

Pharmacokinetic Data

TK comparison of triptolide levels in Calvert and SRI studies—Males and Females

Plasma Triptolide Concentration (ng/ml)

| | Plasma Triptolide Concentration (ng/ml) | | | | | |
|---|---|---|---|---|---|---|
| Time (hrs)> | 0 | 0.25 | 0.5 | 1 | 2 | 24 |
| PG796 (MRx102) | 0 | 11.8 | 10.5 | 3.1 | 5.8 | 0 |
| 0.5 mg/kg (emulsion) | | | | | | |
| Triptolide | 0 | 74.6 | 18.4 | 13.2 | 0 | 0 |
| 0.15 mg/kg (emulsion) | | | | | | |
| PG796 (MRx102) | 0 | 36.4 | 29.1 | 16.7 | 4.11 | 0 |
| 1.5 mg/kg | | | | | | |
| (DMSO/PEG400/PBS) | | | | | | |

MRx102 0.5 mg/kg and Triptolide 0.15 mg/kg are from Calvert study; results are from females
MRx102 1.5 mg/kg is from SRI study; results are from males
SRI study - 3, 4, 8 hrs. triptolide concentration = 0 ng/ml

TK Comparison of Triptolide Levels in Calvert and SRI Studies—Males Only

| | Plasma Triptolide Concentration (ng/ml) | | | | | |
|---|---|---|---|---|---|---|
| Time (hrs)> | 0 | 0.25 | 0.5 | 1 | 2 | 24 |
| PG796 (MRx102) | 0 | 32.5 | 10.9 | 0.7 | 1.0 | 0 |
| 0.5 mg/kg (emulsion) | | | | | | |
| Triptolide | 0 | 59.0 | 14.9 | 3.6 | 0 | 0 |
| 0.15 mg/kg (emulsion) | | | | | | |
| PG796 (MRx102) | 0 | 36.4 | 29.1 | 16.7 | 4.1 | 0 |
| 1.5 mg/kg | | | | | | |
| (DMSO/PEG400/PBS) | | | | | | |

MRx102 0.5 mg/kg and Triptolide 0.15 mg/kg are from Calvert study; results are from males
MRx102 1.5 mg/kg is from SRI study; results are from males
SRI study - 3, 4, 8 hrs. triptolide concentration = 0 ng/ml

Routes of Administration

Although in some embodiments the route of administration is intravenous, other routes include: epicutaneous or topical, intradermal, subcutaneous, nasal, intraarterial, intramuscular, intracardiac, intraosseous infusion, intrathecal, intraperitoneal, intravesical, intravitreal intracavernous injection, intravaginal, and intrauterine.

### Example 2

### Cytotoxicity (MTT) Assay

Test compounds may be dissolved in DMSO at a concentration of 20 mM. Further dilutions may be done in RPMI1640 medium (GIBCO, Rockville, Md.) supplemented with 10% Fetal Calf Serum (HyClone Laboratories, Logan, Utah).

Cytotoxicity of the compounds is determined in a standard MTT assay using Cell Proliferation Kit I (#1 465 007, Roche Diagnostics, Mannheim, Germany). Briefly, human T cell lymphoma (Jurkat) cells (4×10⁵ per well) are cultured for 24 h, in 96-well tissue culture plates, in the presence of serial three-fold dilutions of test compounds or medium containing the same concentration of DMSO as in the test samples at each dilution point. The cultures are then supplemented with 10 μl/well MTT reagent for 4 h and then with 0.1 ml/well solubilizing reagent for an additional 16 h. Optical density at 570 nm (OD₅₇₀) is measured on a ThermoScan microplate reader (Molecular Devices, Menlo Park, Calif.).

### Example 3

### IL-2 Production Assay

Test samples can be diluted to 1 mM in complete tissue culture medium. Aliquots are placed in microculture plates coated with anti-CD3 antibody (used to stimulate the production of IL-2 by Jurkat cells), and serial dilutions are prepared so that the final concentration encompass the range of 0.001 to 10,000 nM in log increments. Cells from an exponentially expanding culture of Jurkat human T cell line (#TIB-152 obtained from American Type Culture Collection, Manassas, Va.) are harvested, washed once by centrifugation, re-suspended in complete tissue culture medium, and diluted to a concentration of 2×10⁶ cells/ml. A volume of 50 μl of Jurkat cells (1×10⁵ cells) is added to wells containing 100 μl of the diluted compounds, 50 μl of PMA (10 ng/ml) is added to each well, and the plates are incubated at 37° C. in a 5% CO₂ incubator. After 24 hours, the plates are centrifuged to pellet the cells, 150 μl of supernatant is removed from each well, and the samples are stored at −20° C. The stored supernatants are analyzed for human IL-2 concentration using the Luminex 100 (Luminex Corporation, Austin, Tex.), Luminex microspheres coupled with anti-IL-2 capture antibody, and fluorochrome-coupled anti-IL-2 detection antibody. The data are expressed as pg/ml of IL-2.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A composition for intravenous administration of an emulsion comprising triptolide or a triptolide derivative having a clogP of 0.5 or higher, the emulsion comprising (a) 15 to 45% by weight lipid, (b) 0 to 50% by weight of a medium chain triglyceride, (c) 0.5 to 3% by weight phospholipid, (d) 0 to 5% by weight of glycerin, (e) 0.1 to 0.3% by weight of a sodium cholate, (f) about 50 to 60% by weight water, and (g) about 0.5 to about 3 mg/mL triptolide or a triptolide derivative.

2. The composition of claim 1, wherein the 15 to 45% by weight lipid is a lipid selected from the group consisting of soybean oil, castor oil, corn oil, cottonseed oil, olive oil, peanut oil, peppermint oil, safflower oil, sesame oil, coconut oil or palm seed oil.

3. The composition of claim 1, wherein the medium chain triglyceride is 20% by weight and is selected from the group consisting of glyceryl trioctanoate, glyceryl trihexanoate, glyceryl triheptanoate, glyceryl trinonanoate and glyceryl tridecanoate.

4. The composition of claim 1, wherein the phospholipid is selected from the group consisting of hydrogenated soy phosphatidylcholine, distearoylphosphatidylglycerol, L-alpha-dimyristoylphosphatidylcholine and L-alpha-dimyristoylphosphatidylglycerol.

5. The composition of claim 1, wherein the glycerin is selected from the group consisting of polyethylene glycol 300, polyethylene glycol 400, ethanol, propylene glycol, N-methyl-2-pyrrolidone, dimethylacetamide, and dimethylsulfoxide.

6. The composition of claim 1, wherein the sodium cholate is selected from the group consisting of sodium taurocholate, sodium tauro-β-muricholate, sodium taurodeoxycholate, sodium taurochenodeoxycholate, sodium glycocholate, sodium glycodeoxycholate and sodium glycochenodeoxycholate.

7. The composition of claim 1, comprising a triptolide derivative selected from the group consisting of compounds according to structure I.

8. The composition of claim 1, comprising a triptolide derivative selected from the group consisting of compounds according to structure II.

9. The composition of claim 1, comprising a triptolide derivative selected from the group consisting of compounds according to structure III.

10. The composition of claim 1, comprising a triptolide derivative selected from the group consisting of compounds according to structure IV.

11. A method of effecting immunosuppression, immunomodulation or inhibiting cell proliferation comprising administering to a subject in need of such treatment an effective amount of a composition of claim 1.

12. A method of inducing apoptosis in a cell, comprising contacting said cell with an effective amount of a composition of claim 1.

13. A composition for oral administration of an emulsion comprising triptolide or a triptolide derivative having a clogP of 0.5 or higher, the emulsion comprising (a) 15 to 45% by weight lipid, (b) 0 to 50% by weight of a medium chain triglyceride, (c) 0.5 to 3% by weight phospholipid, (d) 0 to 5% by weight of glycerin, (e) 0.1 to 0.3% by weight of a sodium cholate, (f) about 50 to 60% by weight water, and (g) about 0.5 to about 3 mg/mL triptolide or a triptolide derivative.

14. A composition for intravenous administration of an emulsion comprising triptolide or a triptolide derivative having a clogP of 0.5 or higher, the emulsion comprising (a) 15 to 45% by weight lipid, (b) 0 to 95% by weight of a medium chain triglyceride, (c) 0.5 to 3% by weight phospholipid, (d) 0 to 5% by weight of glycerin, (e) 0.1 to 0.3% by weight of a sodium cholate, and (f) about 0.5 to about 3 mg/mL triptolide or a triptolide derivative; wherein an aqueous solution is added prior to administration.

15. A composition for oral administration of an emulsion comprising triptolide or a triptolide derivative having a clogP of 0.5 or higher, the emulsion comprising (a) 15 to 45% by weight lipid, (b) 0 to 95% by weight of a medium chain triglyceride, (c) 0.5 to 3% by weight phospholipid, (d) 0 to 5% by weight of glycerin, (e) 0.1 to 0.3% by weight of a sodium cholate, and (f) about 0.5 to about 3 mg/mL triptolide or a triptolide derivative; wherein an aqueous solution is added prior to administration.
